(19) United States
(12) Patent Application Publication
(10) Pub. No.: US 2005/0142096 A1
(43) Pub. Date: Jun. 30, 2005

(54) Product for and method of controlling odor in open waste water treatment environments

(76) Inventor: Paul Wegner, San Carlos, CA (US)

Correspondence Address: BENASUTTI, P.A., 17294 BERMUDA VILLAGE DRIVE, BOCA RATON, FL, 33487, US

(21) Appl. No.: 11/067,364

(22) Filed: Feb. 25, 2005

Related U.S. Application Data
(63) Continuation-in-part of application No. 10/058,548, filed on Jan. 28, 2002.
(60) Provisional application No. 60/265,147, filed on Jan. 29, 2001.

(51) Int. Cl. A61L 9/00; A61L 9/01

(52) U.S. Cl. 424/76.1

(57) ABSTRACT

A product for substantially eliminating existing foul odors (such as those associated with materials such as hydrogen sulfide) and substantially preventing the production of new foul odors in matter, in open treatment environments is disclosed and comprises calcium nitrate and calcium carbonate.

### CROSS-REFERENCE TO RELATED APPLICATIONS

This application is a continuation-in-part of my prior co-pending application, entitled PRODUCT FOR AND METHOD OF CONTROLLING ODOR, Ser. No. 10/058,548, filed Jan. 28, 2002, which was a continuation-in-part of my prior application ODOR CONTROL METHOD, Ser. No. 60/265,147, filed Jan. 29, 2001; the disclosures of both of these applications are incorporated herein by reference as if fully set forth; and I claim the benefit of the filing dates of both applications.

### BACKGROUND OF THE INVENTION

1. Technical Field

This invention relates to the control of odors, and more particularly, to the immediate odor removal and prevention of the production of additional odor by waste materials over extended periods of time in open waste water treatment environments.

2. Background Art

In the prior art, the collection of biological materials such as human and animal wastes and the processing of those materials in open waste water treatment environments, such as, lagoons creates foul odors. The waste water generated from the cleaning of animal containment areas, food processing plants, slaughter houses, and portable toilets, and the like contain high levels of organic material, contains odoriferous materials that produce odors by the action of bacteria.

USFilter Distribution Group has a patent (U.S. Pat. No. RE36,651) that discloses the use of nitrates to remove foul odors. It states that aerobic bacteria must be present, nitrates must be present, and a certain amount of time is required before the odors can be removed. The body of the patent describes how aerobic bacteria use nitrates as an oxygen source to oxidize odoriferous compounds, such as mercaptans. The patent acknowledges that the biological oxidation of odiferous material is slow and requires a significant amount of time. It also acknowledges that the prior art teaches the use of nitrates to inhibit the production of odors from anaerobic bacteria.

The Proctor and Gamble patent (U.S. Pat. No. 6,287,550) teaches the inhibition of odors through the use of three measures:
1) using materials that inhibit the formation of odor by various bacteria, enzymes, or pH buffering agents;
2) using materials that absorb odors; and
3) using materials that absorb liquids.

The patent discloses the use of fresheners that must contain perfume in combination with materials that inhibit the formation of odors. The only focuses on the inhibition of odor formation is by the use of inhibitors of bacteria, enzymes, or buffering agents. It does not teach the use of nitrates, or materials such as metal oxides that favor the colonization of aerobic bacteria over anaerobic bacteria by providing an aerobic environment.

History has demonstrated that the complete inhibition or elimination of anaerobic bacteria over extended periods of time in an anaerobic environment is limited and unreliable at best in open systems; such as ponds and lagoons. Even the use of proven potent disinfectants such as chlorine, ultraviolet light, or ozone, dissipate over time and the organic waste is soon colonized with anaerobic bacteria that are present in the air or in the waste materials themselves.

Canned goods are an example of an anaerobic environment being maintained over extended periods of time, without the production of any foul odors. This is achieved with heat and no chemicals. In this case, the bacteria present are destroyed and all subsequent bacteria are excluded from the environment to prevent the formation of foul odors.

In an open treatment environment, however, where bacteria are already present or subsequently inoculate the medium, the colonization of anaerobic bacteria is favored over aerobic bacteria as long as anaerobic conditions prevail. When anaerobic conditions prevail, aerobic bacteria are displaced by anaerobic bacteria. When aerobic conditions prevail, anaerobic bacteria are displaced by aerobic bacteria.

The prior art fails to adequately address the need for creating an environment that favors aerobic bacteria, by avoiding the addition of organic compounds (which contribute to the oxygen demand) or the addition of agents such as nitrates to increase the supply of biologically available oxygen. The primary focus is on the inhibition of odor causing bacteria or enzymes.

The USFilter patent fails to provide for the instant removal of foul odors, while the Proctor and Gamble patent fails to provide agents that provide a significant improvement in the supply of biologically available oxygen.

Other prior art includes the use of hydrogen peroxide and nitrate to destroy odors; but this technique is not effective for long term odor control.

### DISCLOSURE OF THE INVENTION

### SUMMARY OF THE INVENTION

I have discovered a product for substantially eliminating existing foul odors (such as those associated with materials such as hydrogen sulfide) and substantially preventing the production of new foul odors in matter, in open treatment environments. This product comprises calcium nitrate and calcium carbonate.

The product is applied by mixing it with the incoming waste water being introduced into the open environment; such as, by introducing it into the liquid at the lift station.

### DESCRIPTION OF THE PREFERRED EMBODIMENTS

I have invented a product for substantially eliminating existing odors and substantially preventing the production of new odors in waste matter in open treatment environments, comprising in combination, calcium nitrate and calcium carbonate.

When these chemicals are combined with waste materials in lagoons, the bacteria which feed on them prevent the system from going anaerobic; thus ensuring that the system is aerobic.

The amount of nitrates required is a function of the total amount organic materials present, biological oxygen demand, rate of oxygen consumption, and the length of time that aerobic conditions need to prevail. The amount of nitrates required increases to makeup for the oxygen deficiency required for a given length of odor control time. The longer the odor control time, the greater the amount of nitrates required up to the maximum amount. The maximum amount of nitrate required is approximately equal to the amount required to convert all the organic matter into carbon dioxide, nitrogen, and sulfates in an oxygen free atmosphere. This is many times larger than the amount required to oxidize existing foul odors.

Once oxygen levels are insufficient to provide aerobic conditions, the risk of odor production increases. The amount of hydrogen sulfides or mercaptans present has very little to do with the practical estimate of nitrates required for the management of foul odors, except, when no organic matter is present. In the vast majority of organic waste odor control situations, the nitrate requirements are dictated by biological oxygen demand, rate of oxygen consumption, and length of odor control time required. Furthermore, governmental requirements strictly control the amount of nitrates that can be discharged in the potable water emanating from a waste water treatment plant.

I have found that the preferred amount of calcium nitrate 0.5% by weight of waste water solids. This, when combined with 0.5% calcium carbonate is sufficient to keep a lagoon aerobic.

Actual ranges of percent are 1% to 0.1%.

The above chemical mixtures can be applied dry or with water as a carrier.

A description of the methods of using the products is as follows:

For septic waste, apply 8 pounds of calcium nitrate and 8 pounds of calcium carbonate to 1000 pounds of waste.

For a one million gallon cattle manure lagoon; receiving 60,000 gallons per day, apply 5 to 10 pounds of calcium nitrate and 5 to 10 pounds of calcium carbonate, per day.

It can also be introduced as a spray or merely dumped into the lagoon. The lagoon may be checked once a week. Sufficient nitrate levels are indicated when the presence of green algae is seen on the surface.

The calculation of pounds is as follows:

8.4 pounds per gallon times the number of gallons Thus for 1000 gallons, this would equal 8400 pounds.

X %=# of pounds necessary to treat.

Septic waste may be, for example, 100,000 gallons per year. Thus, 500 gallons per day would be mixed with 60,00 gallons of water and 5 pounds of each chemical per day.

The same would be used in bio-solid sludge from large waste water treatment plants; that is, 100,000 gallons per year times 0.5%.

Situations.

1. In controlling odors in a liquid waste such as septic tanks, portable toilets, or organic waste lagoons, both agents should be water soluble; such as, a mixture of calcium nitrate and calcium carbonate.

2. In controlling odors on surfaces, spray is effective.

My new product and my new methods are as follows:

A product for substantially eliminating existing odors and substantially preventing the production of new odors in matter, comprising in combination calcium nitrate and calcium carbonate.

The amount of nitrate may be proportional to the amount required to maintain aerobic conditions over the required time period; as dictated by observation.

If the product is used in a lagoon, the nitrate ion should be 5 to 200 parts per million.

A method of substantially eliminating existing odors and substantially preventing the production of new odors in matter, comprising applying a product to said matter, which product comprises a combination of calcium nitrate and calcium carbonate. The method may comprise products which additionally comprise water and are applied as a spray.

This keeps control of the odor from brown anaerobic systems to a green aerobic system, as a result of the nitrate allowing the aerobic matter to become dominant. The calcium gets rid of the immediate odor.

This procedure does not interfere with the accepted operational parameters of the processing plant; that is, the plant can still meet the chemical oxygen demand (COD) and the biological oxygen demand (BOD) leaving the plant and at the same time meet the nitrogen demand.

It will also meet the suspended solids requirements, that is, it can treat odor without interfering with the quality of the water leaving the plant. By way of further example, the required nitrate limit for drinking water in the State of California is 10 mg/l. That standard will be met with the use of my process. That is, I have invented method of treating waste water in lagoons to substantially eliminate existing odors and substantially prevent the production of new odors in the waste water which has waste solids; which method meets a nitrate limit for drinking water of 10 mg/l, comprising combining calcium nitrate and calcium carbonate with the waste solids in the following proportion of calcium nitrate and calcium carbonate: 0.5% by weight of calcium nitrate and 0.5% by weight calcium carbonate to the weight of waste solids.

1. A product for substantially eliminating existing odors and substantially preventing the production of new odors in waste matter having waste solids in open environments, comprising in combination, calcium nitrate and calcium carbonate;
wherein the proportion of the calcium nitrate and calcium carbonate is as follows: 0.5% by weight of calcium nitrate and 0.5% by weight calcium carbonate to the weight of waste solids.

2. The product of claim 1 wherein the proportion by weight of calcium nitrate and calcium carbonate to the weight of waste solids is in the range of 1% to 0.1%.

3. A method of substantially eliminating existing odors and substantially preventing the production of new odors in waste matter having waste solids in open environments, comprising combining calcium nitrate and calcium carbonate with the waste solids;
wherein the proportion of the calcium nitrate and calcium carbonate is as follows: 0.5% by weight of calcium nitrate and 0.5% by weight calcium carbonate to the weight of waste solids.

4. The method of claim 3 wherein the proportion by weight of calcium nitrate and calcium carbonate to the weight of waste solids is in the range of 1% to 0.1%.

5. A method of treating waste water in lagoons to substantially eliminate existing odors and substantially prevent the production of new odors in the waste water which has waste solids; which method meets a nitrate limit for drinking water of 10 mg/l, comprising combining calcium nitrate and calcium carbonate with the waste solids in the following proportion of calcium nitrate and calcium carbonate: 0.5% by weight of calcium nitrate and 0.5% by weight calcium carbonate to the weight of waste solids.
